(19) United States
(12) Patent Application Publication
(10) Pub. No.: US 2020/0027563 A1
(43) Pub. Date: Jan. 23, 2020

(54) Method of Assessing Risk

(71) Applicant: Neumann Diagnostics, Ltd., Budapest (HU)

(72) Inventor: Miklos Nyiri, Budapest (HU)

(21) Appl. No.: 16/530,353

(22) Filed: Aug. 2, 2019

Related U.S. Application Data
(63) Continuation of application No. PCT/GB2018/050298, filed on Feb. 1, 2018.

(30) Foreign Application Priority Data
Feb. 2, 2017 (GB) 1701719.5

(51) Int. Cl. G16H 50/30; G01N 33/574; C12Q 1/6886

(52) U.S. Cl. CPC G16H 50/30; C12Q 2600/154; C12Q 1/6886; G01N 33/57411

(57) ABSTRACT

The application relates to a method of assessing the risk of a patient for cervical pre-cancer or cancer, especially where there is no sign of a malignancy.

### RELATED APPLICATIONS

This application is a continuation of International Application No. PCT/GB2018/050298, which designated the United States and was filed on Feb. 1, 2018, published in English. This application claims priority under 35 U.S.C. § 119 Great Britain, Application No. 1701719.5, filed Feb. 2, 2017. The entire teachings of the above applications are incorporated herein by reference.

The application relates to a method of assessing the risk of a patient for cervical pre-cancer or cancer, especially where there is no sign of a malignancy.

### BACKGROUND OF THE INVENTION

Throughout life, healthy cells in the body divide, grow, and replace themselves in a controlled fashion. Cancer starts when the genes controlling this cellular division malfunction, and cells begin to multiply and grow out of control.

The cervix is the lower part of the uterus. The part of the cervix closest to the body of the uterus is called the endocervix. The part next to the vagina is the exocervix (or ectocervix). The two main types of cells covering the cervix are squamous cells (on the exocervix) and glandular cells (on the endocervix). The place where these two cell types meet is called the transformation zone. Most cervical cancers start in the transformation zone. These cells gradually develop precancerous changes that turn into cancer. The terms to describe these precancerous changes, include cervical intraepithelial neoplasia (CIN) in the field of histology, and squamous intraepithelial lesion (SIL) in the field of cytology, and dysplasia.

Cervical cancer is a malignancy of the cervix. Most scientific studies have found that human papillomavirus (HPV) infection is responsible for virtually all cases of cervical cancer. Worldwide, cervical cancer is the third most common type of cancer in women. However, it is much less common in countries where the routine use of Pap smears is widespread. Cervical cancers and cervical precancers are classified by morphology. There are two main types of cervical cancer: squamous cell cancer and adenocarcinoma, named after the type of cell that becomes cancerous. Squamous cells are the flat skin-like cells that cover the outer surface of the cervix (the ectocervix). About 80% to 90% of cervical cancers are squamous cell carcinomas. Adenomatous cells are gland cells that produce mucus in the endocervix. The cervix has these gland cells scattered along the inside of the passageway that runs from the cervix to the womb. Adenocarcinoma is a cancer of these gland cells.

Less commonly, cervical cancers have features of both squamous cell carcinomas and adenocarcinomas. These are called adenosquamous carcinomas or mixed carcinomas.

Cervical cancer may present with abnormal vaginal bleeding or discharge. Other symptoms include weight loss, fatigue, pelvic pain, back pain, leg pain, single swollen leg, and bone fractures. However, symptoms may be absent until the cancer is in its advanced stages. Undetected, pre-cancerous changes can develop into cervical cancer and spread to the bladder, intestines, lungs, and liver.

Although cervical cancers start from cells with precancerous changes (precancers), only some women with precancers of the cervix will develop cancer. Precancers are classified as cervical intraepithelial neoplasia (CIN) according to mechanical morphological classification system numbered from 1 to 3 with increasing severity. The cervical precancer and cancer grades are cervical intraepithelial neoplasia grade 1 (CIN1), cervical intraepithelial neoplasia grade 2 (CIN2) and cervical intraepithelial neoplasia grade 3 (CIN3) and cervical cancer. The change from cervical precancer to cervical cancer usually takes several years, but it can happen in less than a year. For most women, precancerous cells will disappear without any treatment. Still, in some women precancers turn into true (invasive) cancers. Treating all precancers can prevent almost all true cancers.

Cervical cancer is the easiest female cancer to prevent, with regular screening tests and follow-up. The screening tests can help prevent cervical cancer or detect it early. Several diagnostic tests and/or screening tests are used to rule out or confirm cervical precancer and cancer. The methods include a cytological approach of taking cervical exocervical and endocervical cells by brush like device (e.g. a Pap smear) and identify the morphological changes associated with the different cervical precancer and cancer pathological categories; or a histological approach of taking intact tissue samples (biopsy) and looking for morphological changes (histology). Both these approaches require visual analysis by a skilled person. While an effective screening tool, the Pap smear is an invasive procedure, and is incapable of offering a final diagnosis. Diagnosis of cervical cancer must be confirmed by surgically removing tissue from the cervix (colposcopy, or cone biopsy), which may also be a painful procedure, and one which causes the patient great discomfort. Recently different methods have been developed which have greater diagnostic accuracy using the concurrent detection of protein in these samples.

All of these methods are not fully satisfactory for the proposed usage. An improved method is needed to screen for cervical precancer and cancer. Ideally these methods should be automatic, eliminating the reliance on visual analysis, whilst still providing a high level of sensitivity and specificity. This would make the test accessible in areas such as developing countries where cytopathology methods are not widely available.

### SUMMARY OF THE INVENTION

The invention relates to a screening method to identify patients who are likely to develop cervical pre-cancer or cancer. Patients identified as being at risk can be monitored more closely and/or sent for further investigations or treatment.

The present invention provides a method of identifying a patient at risk of developing cervical pre-cancer or cervical cancer comprising:
(a) Measuring the level of methylation of a cervical cancer marker in a sample obtained from the patient; and
(b) Determining the level of risk based on the level of methylation and the patient's age.

The method is useful in women aged 20-60 years old, such as 30-50 years. It is especially useful in subjects over the age of 30 years, in particular age 35 and above. The patient is a human subject.

“Cervical cancer marker” as used herein refers to a marker which has an increased level of methylation in patients with cervical cancer. Such markers are well known in the art. Preferably the cervical cancer marker is selected from POU4F3 (as described in Chen et al (2014) Journal of international Cancer 135(1):117-27) or HS3ST2 (heparan sulfate-glucosamine 3-sulfotransferase 2), both of which show high levels of methylation in cancerous tissue. Most preferably the marker is POU4F3, as this demonstrates a high level of methylation in positive samples, and the greatest difference between positive and negative samples i.e. cancerous and non-cancerous states. The sequence for HS3ST2 is identified as Gene ID: 9956 as updated on 27 Jan. 2018 (https://www.ncbi.nlm.nih.gov/gene/9956).

The level of methylation of a single marker can be measured. Alternatively, the level of methylation of a plurality of markers can be measured. Preferably the level of methylation of POU4F3 and/or HS3ST2 is measured.

A “cervical precancer”, which is also called an intraepithelial lesion, is an abnormality in the cells of the cervix that could develop into cervical cancer. There are two main types of cervical cells, squamous and glandular, and abnormalities can occur in either type. As defined herein, “cervical precancer” includes grades cervical intraepithelial neoplasia grade 1 (CIN1), cervical intraepithelial neoplasia grade 2 (CIN2) and cervical intraepithelial neoplasia grade 3 (CIN3).

“Cervical Cancer” is a malignancy of the cervix. Types of malignant cervical tumors include squamous cell carcinoma, adenocarcinoma, adenosquamous carcinoma. As defined herein, the term “cervical cancer” includes Stage I, Stage II, Stage III and Stage IV cervical cancer, as defined by the TNM staging system.

“Risk” in the context of the present invention, relates to the probability that an event will occur over a specific time period, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of lower risk cohorts, across population divisions (such as tertiles, quartiles, quintiles, or deciles, etc.) or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event).

“Risk evaluation,” or “evaluation of risk” in the context of the present invention encompasses making a prediction of the probability, odds, or likelihood that an event or disease state may occur, and/or the rate of occurrence of the event or conversion from one disease state to another, i.e., from a normal condition to cancer or from cancer remission to cancer, or from primary cancer occurrence to occurrence of a cancer metastasis. Risk evaluation can also comprise prediction of future clinical parameters, traditional laboratory risk factor values, or other indices of cancer results, either in absolute or relative terms in reference to a previously measured population. Such differing use may require mathematical algorithms, and/or cut-off points, but be subject to the same aforementioned measurements of accuracy and performance for the respective intended use.

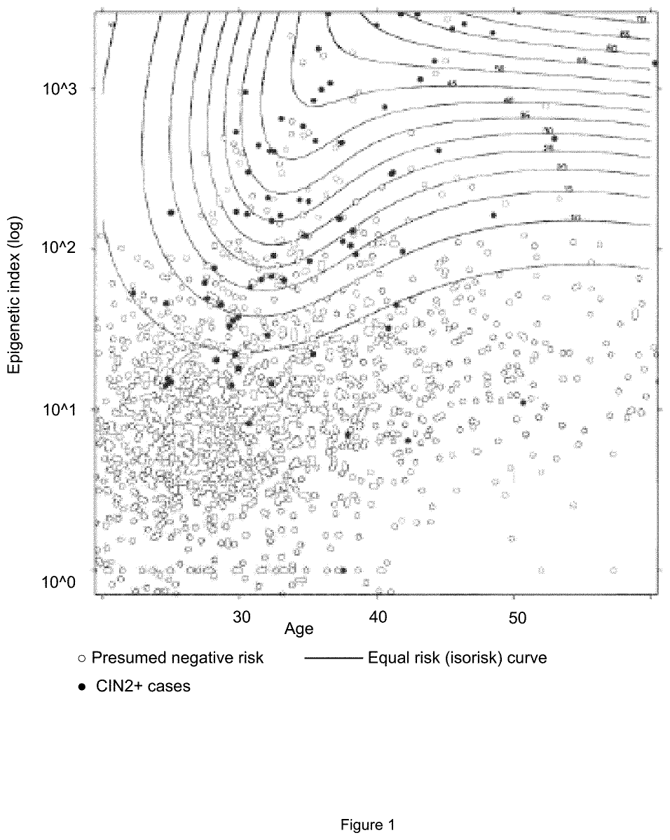

The level of risk can be determined by correlating the level of marker methylation with the patient's age, utilising an algorithm for a risk curve, such as that shown in FIG. 1. The level of risk can be expressed as a % likelihood of developing cervical precancer or cancer.

The risk curve is generated by measuring the level of methylation in samples obtained from healthy controls as well as patients who have been diagnosed with cervical cancer. The ratio of confirmed pre-cancer or cancer cases to presumed histology negatives are then plotted according to epigenetic result and age of the patient. The generalized additive model (GAM) model and logistic regression are then used to create the isorisk map.

A “sample” is any sample derived from a subject that contains nucleic acids such as RNA and/or DNA. It may include a single cell or multiple cells or fragments of cells or an aliquot of body fluid, taken from the subject. The sample may be selected from the group consisting of a body fluid, a cell or population of cells, or a tissue from the subject. For example, the cell is a cervical cell, or a rare circulating tumor cell or circulating endothelial cell found in the blood. Cell and tissue samples can be obtained by means including biopsy, needle aspirate, lavage sample, scraping, cervical Pap smear, surgical incision or intervention or other means known in the art. A suitable sample may be a cervical brush sample which is prepared as follows: Samples are taken from the ectocervical and endocervical cells and immediately fixed in alcoholic buffered solutions, such as Thin Prep PreservCyt sampling, preservative liquid. “Body fluid” of a subject includes blood, urine, spinal fluid, lymph, mucosal secretions, haemolymph or any other body fluid known in the art for a subject. The blood sample can include blood discharged during menstruation obtained from a tampon or other sanitary item.

Diagnosis of cervical precancer and cancer is made, for example, from the method of the invention, optionally in combination with any one or more of the following procedures: a medical history, a Pap smear, and biopsy procedures (including cone biopsy and/or colposcopy). In particular, the method of the invention can be carried out on samples from patients where no sign of malignancy has been identified using conventional methods i.e. those patients who did not get a severe dysplasia diagnosis using conventional methods. The method of the present application can establish an individual risk for each patient for cervical pre-cancer or cancer. This can potentially detect underlying disease, and so lead to earlier treatment of the condition and an increased chance of survival. The method of the present application can be used to increase the sensitivity of conventional Pap testing, without impacting specificity. The method of the invention was able to detect severe cases up to 12 months earlier that by using a Pap smear.

A subject can also include those who are suffering from, or at risk of developing cervical cancer or a condition related to cervical cancer, such as those who exhibit known risk factors for cervical cancer or conditions related to cervical cancer. Known risk factors for cervical cancer include but are not limited to: human papillomavirus (HPV) infection, smoking, HIV infection, chlamydia infection, dietary factors, the use of oral contraceptives, multiple pregnancies, the use of the hormonal drug diethylstilbestrol (DES) and a family history of cervical cancer.

The level of methylation of the marker is determined from the nucleic acid within the sample. Nucleic acids, RNA and/or DNA, are purified from cells, tissues or fluids of the test population of cells. RNA is preferentially obtained from the nucleic acid mix using a variety of standard procedures (e.g. RNA Isolation Strategies, pp. 55-104, in RNA Methodologies, A laboratory guide for isolation and characterization, 2nd edition, 1998, Robert E. Farrell, Jr., Ed., Academic Press), or using a filter-based RNA isolation system such as that from Ambion (RecoverAll Total Nucleic Acid Isolation Kit).

Methods of measuring the level of methylation of the marker are well known in the art. Suitable methods include Methylation-Specific PCR (MSP), bisulfite sequencing including whole genome bisulfite sequencing (also known as BS-Seq), ChIP-on-chip assays, Methylated DNA immunoprecipitation (MeDIP), pyrosequencing, quantitative analysis of methylated alleles (QAMA) and restriction enzyme based methods such as the HELP assay, Restriction landmark genomic scanning and Methyl Sensitive Southern Blotting. Preferably the level of methylation is determined by methylation sensitive PCR.

The methods of the invention may require amplification of the nucleic acid sample from the subject, and this can be done by techniques known in the art, such as PCR (see PCR Technology: Principles and Applications for DNA Amplification (ed. H. A. Erlich, Freeman Press, NY 1992; PCR Protocols: A Guide to methods and Applications (eds. Innis et al., Academic press, San Diego, Calif. 1990); Manila et al., Nucleic Acids Res. 19 4967 (1991); Eckert et al., PCR Methods and Applications 117 (1991) and U.S. Pat. No 4,683,202. Other suitable amplification methods include ligase chain reaction (LCR) (Wu et al., Genomics 4 560 (1989); Landegran et al., Science 241 1077 (1988)), transcription amplification (Kwoh et al., Proc Natl Acad Sci USA 86 1173 (1989)), self sustained sequence replication (Guatelli et al., Proc Natl Acad Sci USA 87 1874 (1990)) and nucleic acid based sequence amplification (NASBA). The latter two methods both involve isothermal reactions based on isothermal transcription which produce both single stranded RNA and double stranded DNA as the amplification products, in a ratio of 30 or 100 to 1, respectively. In particular these methods can be carried out before or after bisulfite conversion. Sodium bisulfite is reacted with DNA to convert unmethylated cytosines of CpG dinucleotides to uracil or UpG. Methylated cytosines are not converted. This allows the methylated sites to be detected.

In a preferred embodiment the method comprises

(a) obtaining a sample of nucleic acid from a cervical cell from a subject;

(b) treating the nucleic acid obtained with sodium bisulfite; and

(c) measuring the level of methylation of a cervical cancer marker within the treated nucleic acid;

(d) determining the level of risk of developing cervical pre-cancer or cancer based on the level of methylation in combination with the age of the subject.

Where it is desirable to analyses multiple samples simultaneously, it may be preferable to use arrays such as those described in WO95/11995. The array may contain a number of probes, each designed to identify the genes or any combination of two or more of the genes from a sample. As the methods rely upon the marker methylation level as opposed to visual analysis of the cells samples, it can be automated. It also reduces the likelihood of mis-diagnosis due to human error.

The methods disclosed herein may be applied to cells of humans, mammals or other organisms without the need for undue experimentation by one of ordinary skill in the art because most cells contain nucleic acids, and it is known in the art how to extract RNA/DNA from different types of cells.

The present invention also relates to the use of the level of cervical cancer marker methylation, in combination with a patients age, to determine the level of risk of developing cervical cancer or pre-cancer.

All references referred to herein are incorporated in their entirety by reference.

The invention will be described in the examples below with reference to the following figures:

FIG. 1 shows an isorisk curve map for POU4F3 showing the ratio of confirmed pre-cancer or cancer cases to presumed histology negatives plotted according to epigenetic result and age of the patient. 1,595 data points representing the epigenetic test result (vertical axis, log of index value) and the age (horizontal axis) of HPV-positive women tested in the clinical trial were plotted in 2 groups: red dots are cases with a confirmed dysplasia or cancer diagnosis; blue dots are presumed histology negative samples. The isorisk curves show the shifting ratio of the 2 groups. Background colour indicates the standard error of the risk level estimate.

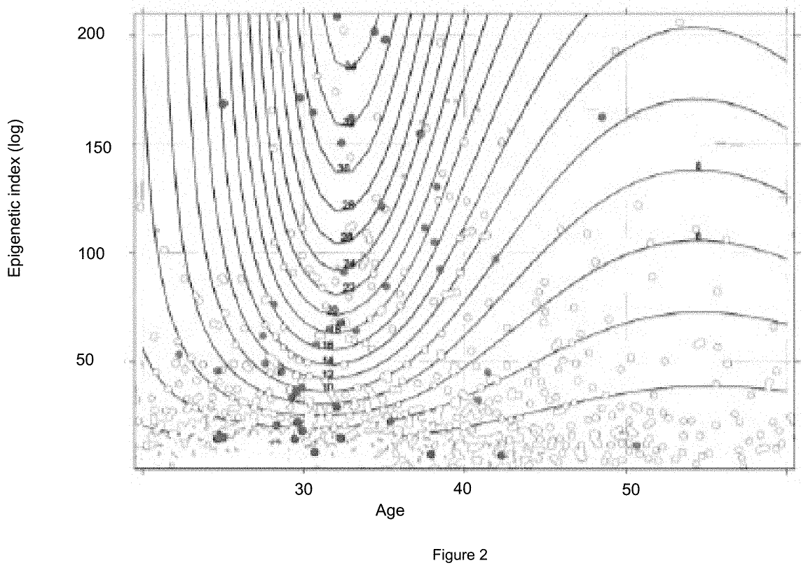

FIG. 2 shows an expanded view of FIG. 1 at lower levels of methylation.

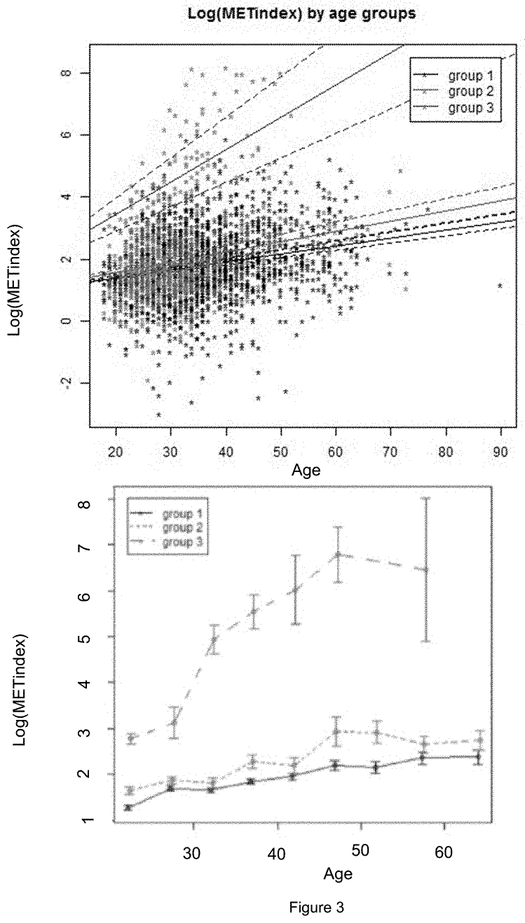

FIG. 3 shows a comparison of readings from three groups of patients generated with POU4F3 marker.

Group 1 (N=1855): HPV− & CITO− & CIN2− (i.e. do not have cervical cancer; healthy controls)

Group 2 (N=712): HPV+ & (CITO≠ASCH|HSIL|CIS|CC) & CIN2− (Give a negative result on traditional smear test, but may be at risk of developing cervical cancer as have HPV infection)

Group 3 (N=59): CIN2+ (i.e., Histologically confirmed to have cervical cancer)

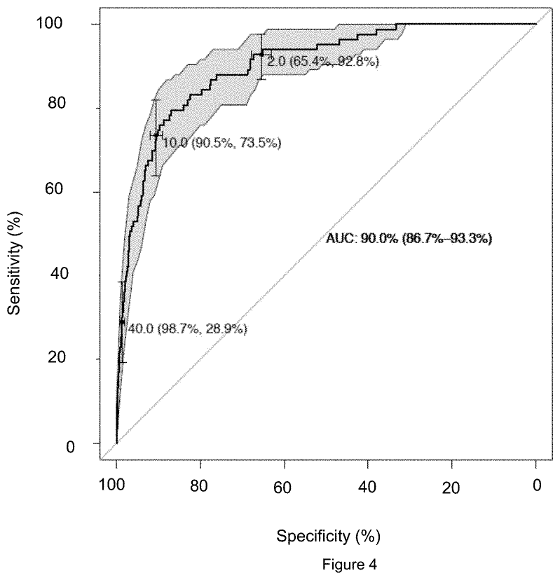

FIG. 4 shows a ROC curve generated with POU4F3 marker indicating that the test of the invention is highly specific and sensitive. The cut-off points at 2%, 10%, and 40% level of risk for CIN2+ are shown. These correlate with the threshold values provided by the National Cancer Institute. Patients falling below the 2% threshold do not require follow up treatment; Patients falling between 2% and 10% require follow up treatment; Patients falling between 10% and 40% require colposcopy; Patients with a result over 40% threshold require treatment even if there is no sign of a lesion.

### EXAMPLE

1,595 clinical samples (Rovers cervical brush), all HPV positives, aged between 20 and 60, collected in a multicentre clinical trial conducted in Hungary were tested. Including HPV negatives over 6,000 women were enrolled.

Samples were bisulfate converted, and tested with Taqman reaction using the primers and probes described in Chen et al (2014) “Methylomics analysis identifies epigenetically silenced genes and implies an activation of β-catenin signaling in cervical cancer” International Journal of Cancer 135(1):117-27 to determine the level of methylation.

The generalized additive model (GAM) model and logistic regression was used to create the isorisk map shown in FIGS. 1 and 2.

The results were further verified by comparing three groups of patients.

Group 1 (N=1855): HPV− & CITO− & CIN2− (i.e. do not have cervical cancer; healthy controls)

Group 2 (N=712): HPV+ & (CITO ASCH|HSIL|CIS|CC) & CIN2− (Give a negative result on traditional smear test, but may be at risk of developing cervical cancer as have HPV infection)

Group 3 (N=59): CIN2+ (i.e. Histologically confirmed to have cervical cancer)

These results show that the level of general methylation increases with age, but that the increase with age is far greater in patients with cervical cancer.

The sensitivity and specificity of the method was assessed and a ROC curve generated. In HPV-positive women aged 25 years or older sensitivity for severe cervical dysplasia or worse condition at baseline was 91.94% (95% CI 82.17-97.33) meanwhile specificity in the same group was 74.35% (95% CI 71.78-76.81). (See FIG. 4)

The level of methylation alone is insufficient to determine the level of risk. This must be used in combination with the patient age. For example, the methylation level could be 50 for in two patient samples. However, the ages of the patients are 32 and 52. Accordingly their calculated risk is 13% (95% CI 0.091 to 0.19) and 2.9% (95% CI 0.012 to 0.067) respectively. These results are significantly different.

1. A method of identifying a patient at risk of developing cervical pre-cancer or cervical cancer comprising:
(a)Measuring the level of methylation of a cervical cancer marker in a sample obtained from the patient; and
(b) Determining the level of risk based on the level of methylation and the patient's age.

2. The method of claim 1 wherein the cervical cancer marker is selected from POU4F3 or HS3ST2.

3. The method of claim 1 wherein the patient is over 30 years of age.

4. The method of claim 1 wherein the level of risk is determined by correlating the level of marker methylation with the patients' age using the isorisk curve shown in FIG. 1 or FIG. 2.

5. The method of claim 1 comprising the steps of
(a) obtaining a sample of nucleic acid from a cervical cell from a subject;
(b) treating the nucleic acid obtained with sodium bisulfite; and
(c) measuring the level of methylation of a cervical cancer marker within the treated nucleic acid; and
(d) determining the level of risk of developing cervical pre-cancer or cancer based on the level of methylation in combination with the age of the subject.

6. The use of the level of methylation of a cervical cancer marker, in combination with a patients age, to determine the level of risk of developing cervical cancer or pre-cancer.
